(19) United States
(12) Patent Application Publication
(10) Pub. No.: US 2023/0158099 A1
(43) Pub. Date: May 25, 2023

(54) STABLE READY TO DILUTE FORMULATIONS OF CARFILZOMIB

(71) Applicant: KASHIV BIOSCIENCES, LLC, Piscataway, NJ (US)

(72) Inventors: Paras Rasiklal VASANANI, Ahmedabad (IN); Sandip Pareshbhai MEHTA, Ahmedabad (IN)

(21) Appl. No.: 17/919,171

(22) PCT Filed: Apr. 15, 2021
(86) PCT No.: PCT/IB2021/053118

§ 371 Date: Oct. 14, 2022

(30) Foreign Application Priority Data
Apr. 15, 2020 (IN) 202021016383
Nov. 12, 2020 (IN) 202021049395

(51) Int. Cl. A61K 38/07; A61K 9/00; A61K 47/22; A61K 47/10; A61K 47/26; A61K 47/12; A61K 47/18; A61K 9/08

(52) U.S. Cl. CPC A61K 38/07; A61K 9/0019; A61K 47/22; A61K 47/10; A61K 47/26; A61K 47/12; A61K 47/18; A61K 9/08

(57) ABSTRACT

The invention provides room temperature stable ready to dilute injectable formulation comprising carfilzomib or it pharmaceutically acceptable derivatives. The invention further provides methods for treating patients with multiple myeloma by administering room temperature stable ready to dilute injectable formulations comprising carfilzomib or its pharmaceutically acceptable derivatives.

### FIELD OF THE INVENTION

The invention is directed to room temperature stable injectable formulations of carfilzomib or its pharmaceutically acceptable derivatives thereof in the form of ready to dilute solution and concentrates. Further the invention is directed to a method for treating patients with relapsed or refractory multiple myeloma which includes method of administering reconstituted ready to dilute solution comprising mixing of component 1 and component 2 to form reconstituted ready to dilute solution wherein the component 1 is stored at room temperature for at least one month.

### BACKGROUND

Carfilzomib is a selective proteasome inhibitor indicated for the treatment of multiple myeloma.

Carfilzomib is a tetrapeptide epoxyketone proteasome inhibitor that irreversibly binds to the N-terminal threonine-containing active sites of the 20S proteasome, the proteolytic core particle within the 26S proteasome.

Carfilzomib is commercially marketed under the name Kyprolis®. in single dose vials containing 10 mg, 30 mg and 60 mg of the active ingredient. Each vial, in addition to lyophilized carfilzomib, also contains sulfobutylether beta-cyclodextrin, citric acid and sodium hydroxide for pH adjustment.

The problems associated with the commercially available formulation is that the reconstitution of the lyophilized product is complex and cumbersome. Since process of reconstitution is complex which comprises aseptically reconstituting each vial by slowly injecting sterile water for injection through the stopper, directing the water onto the inside wall of the vial in order to ensure less foam formation. If foaming occurs, one has to wait till the foam is settled down and subsides till the solution becomes clear. Also, in reconstitution products it is of great importance to visually inspect the solution before administration and a reconstituted solution that appears to have any discoloration or particulate matter must be discarded. Also, it is known that if there is excess foam then it may lead to loss in potency.

There have been efforts to obtain improved carfilzomib compositions. For instance, substituted cyclodextrin additives have been explored to enhance the solubility of carfilzomib.

As carfilzomib is sensitive to degradation, the development of cost-effective room temperature stable carfilzomib injection is very challenging. There remains a need for improved formulations of carfilzomib having improved ease of manufacture, means of administration, and stability over time, especially when stored under room temperature.

### SUMMARY OF THE INVENTION

It is an object of the invention to provide room temperature stable ready to dilute injectable formulation comprising Carfilzomib or it pharmaceutically acceptable derivatives.

It is another object of the present invention to provide methods for treating patients with multiple myeloma by administering room temperature stable ready to dilute injectable formulations comprising carfilzomib or its pharmaceutically acceptable derivatives.

Further, the object of the present invention is to provide a method for treating patients with relapsed or refractory multiple myeloma which includes method of administering reconstituted ready to dilute solution comprising mixing of component 1 and component 2 to form reconstituted ready to dilute solution followed by diluting the reconstituted ready to dilute solution with infusion media; wherein the component 1 is room temperature stable ready to dilute parenteral formulation of carfilzomib or its pharmaceutically acceptable salts; and component 2 is acidifying agent.

In one aspect of the present invention provides room temperature stable carfilzomib formulation which is stable over a period of at least one month when stored at 25° C.

In one aspect of the present invention provides room temperature stable carfilzomib formulation which is stable over a period of at least one month when stored at 25° C. wherein the formulation does not have total impurity more than 6%, preferably not more than 5%, preferably not more than 4%, preferably not more than 3%, preferably not more than 2%, preferably not more than 1.5%, preferably not more than 1%, preferably not more than 0.5%.

### DETAILED DESCRIPTION OF THE INVENTION

As used in the specification and the appended claims, the singular forms “a,” “an” and “the” include plural referents unless the context clearly dictates otherwise.

As used herein, the “about” term refers to the deviation of ±5% from the said value.

As used herein, the term “optional” or “optionally” means that the subsequently described event or circumstance may or may not occur.

The formulation of the present invention contains “carfilzomib” or its pharmaceutically acceptable derivatives a. The pharmaceutically acceptable derivatives include pharmaceutically acceptable salts, solvates, hydrates, anhydrates, enantiomers, isomers, polymorphs, tautomers or mixture thereof.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, ptoluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

The term “stable formulation” or “stabilized formulation” refers to any preparation of carfilzomib having sufficient physical and chemical stability to allow storage at a convenient temperature, for a reasonable period of time.

As used herein, the term “room temperature” refers to temperature between about 15° C. to about 40° C.

As used herein, the term “carfilzomib impurity” refers to any compound resulting from the chemical degradation of carfilzomib. Exemplary degradation pathways include but not limited to amide and/or epoxide hydrolysis, oxidation, epimerization, and products resulting from oxirane-ring opening with various nucleophiles.

As used herein, the term “ready to dilute” refers to a formulation of carfilzomib or its pharmaceutically acceptable derivatives thereof which can be directly combined with an infusion media (e.g., dextrose solution, water for injection, Ringer's solution, isotonic sodium chloride solution, suitable non-aqueous solvents or any other infusion medium) and then administered to a patient. In some embodiments, the ready to dilute formulation may be provided as a single vial containing the injectable formulation comprising carfilzomib or its pharmaceutically acceptable derivatives. Optionally, the ready to dilute formulation may be further diluted with other suitable excipients before combining with infusion media.

As used herein, the term “component 1” refers to a ready to dilute formulation of carfilzomib or its pharmaceutically acceptable derivatives thereof.

As used herein, the term “component 2” refers to an acidifying agent which is reconstituted with the component 1 to form reconstituted ready to dilute formulation which can optionally be further added or mixed into infusion media. The Component 2 is used in clear solution form or powder form.

As used herein, the term “reconstituted ready to dilute” refers to a formulation of carfilzomib or its pharmaceutically acceptable derivatives thereof obtained after mixing the component 1 and the component 2 before adding into infusion media.

The term “ready to use” refers to any preparation of carfilzomib or its pharmaceutically acceptable derivatives thereof which can be administered to patient directly without any further dilution or processing.

The formulations of the present invention are injectable formulation. The injectable formulation of carfilzomib or its pharmaceutically acceptable derivatives according to present invention may be administered via any route including intramuscular, intravenous, or subcutaneous. Preferably, the injectable formulation of the present invention may be administered intravenously. The formulations of carfilzomib or its pharmaceutically acceptable derivatives thereof can be in the form of liquid concentrates, ready to dilute or ready to use solutions. The injectable formulations may be packaged within a conventional sterile vial or other suitable sterile container.

In one embodiment, the room temperature stable ready to dilute injectable formulation comprising carfilzomib or its pharmaceutically acceptable derivatives contains carfilzomib at concentrations from about 5 mg/mL to about 350 mg/mL. In one embodiment, the concentrations of carfilzomib are in the range from about 10 mg/mL to about 100 mg/mL, or about 15 mg/mL to about 60 mg/mL, or about 10 mg/mL to about 60 mg/mL. In one preferred embodiment, the injectable formulation is the one having carfilzomib at a concentration of about 10 mg/mL or about 60 mg/mL.

In one embodiment, the present invention provides room temperature stable injectable ready to dilute formulation comprising carfilzomib or its pharmaceutically acceptable derivatives thereof and one or more solvent. In one embodiment, the ready to dilute compositions can include one or more solvent selected from ethanol, isopropyl alcohol, benzyl alcohol, propylene glycol, polyethylene glycol, glycerol, dimethylacetamide (DMA), N-methylpyrrolidone, dimethylsulfoxide (DMSO), diethylene glycol monoethyl ethers, caprylocaproyl polyoxyl-8 glycerides, glycofurol, or mixtures thereof. In preferred embodiments the formulations can include ethanol, dimethylacetamide, propylene glycol, polyethylene glycol, or mixtures thereof. In one embodiment, the ratio of the one or more solvent used and the amount of carfilzomib or its pharmaceutically acceptable derivatives can vary from about 100:1 to 8:1.

In one embodiment, the room temperature stable injectable formulation of the present invention may optionally comprise one or more pharmaceutically acceptable excipients, such as a buffer, surfactant, antioxidant and preservative.

The formulation can include one or more pharmaceutically acceptable surfactants. Suitable surfactants include anionic, cationic, amphoteric and non-ionic surfactants, Exemplary non-ionic surfactants include polyethylene oxides, for instance PEG 300 or PEG 400. Pharmaceutically acceptable surfactant for this application include, but are not limited to polysorbate or polyethoxylated castor oil, Polyoxyl 20 stearate, Polyoxyl 35 castor oil, poloxamer, polyoxyethylene sorbitan monoisostearate, polyethylene glycol 40 sorbitan diisostearate, Polyoxyl 40 Hydrogenated castor oil, Polysorbate, Polysorbate 20, Polysorbate 40, Polyoxyl 60 stearate, Polysorbate 80, Polysorbate 60, poloxamer 331, polyoxyethylene fatty acid esters, Polyoxyl 40 castor oil, poloxamer 188, polyoxyethylene polyoxypropylene 1800, oleic acid, Sodium desoxycholate, Sodium lauryl sulfate, Sorbitan monolaurate, Sorbitan monooleate, Sorbitan monopalmitate, Sorbitan trioleate, N-Carbamoyl methoxypolyethylene glycol 2000-1,2-distearol, myristic acid, Steareth, Stearic acid, Polyoxyl 40 stearate, Sucrose stearate,

Tocopherol, Triglyceride synthetic, Trimyristin, Tristearin, magnesium stearate, lecithin, lauryl sulfate, Vitamin E, vitamin E-TPGS, egg yolk phosphatides, docusate sodium, dimyristoyl phosphatidylglycerol, dimyristoyl lecithin, Capryol 90 (propylene glycol monocaprylate), Capryol PGMC (propylene glycol monocaprylate), deoxycholate, cholesterol, Cremophor EL, Propylene glycol alginate, Croval A-10 (PEG 60 almond glycerides), Labrafil 1944 (oleoyl macrogol-6 glycerides), Labrafil 2125 (linoleoyl macrogol-6 glycerides), Labrasol (caprylocaproyl macrogol-8 glycerides), Lauroglycol 90 (propylene glycol monolaurate), Lauroglycol FCC (propylene glycol laurate), calcium stearate, Lecithin Centromix E, Lecithin Centrophase 152, Lecithin Centrol 3F21B, POE 26 glycerin, Olepal isosteariques (PEG-6 isostearate), Plurol diisostearique (polyglycerol-3-diisostearate), Plurol Oleique CC, POE 20 Sorbitan trioleate, Tagat TO (polyoxyethylene glycerol trioleate), or Solutol (macrogol-15 hydroxystearate). In some embodiments, optionally the surfactant can be present in an about 10% to about 90% of the total weight of the ready to dilute formulation, preferably from about 20% to about 80% of the total weight of the ready to dilute formulation, preferably from about 30% to about 60% of the total weight of the ready to dilute formulation.

The formulation can include buffer selected from mixtures of a weak acid and alkali metal salt (e.g., Sodium, potassium) and the conjugate base of the weak acid. Suitable buffers include, for example, buffers selected from the group consisting of citric acid, acetic acid, maleic acid, phosphoric acid, succinic acid, tartaric acid, ascorbic acid, benzene sulfonic acid, oxalic acid, fumaric acid, gluconic acid, malic acid, methane sulfonic acid, p-toluenesulfonic acid, naphthalene sulfonic acid, lacturonic acids, lactic acid, lactobionic acid, edetic acid, gentisic acid, meta phosphoric acid, nitric acid, pentetic acid, glycolic acid as well as the counter ion salts thereof.

The formulation can include one or more antioxidant selected from butylated hydroxytoluene, butylated hydroxy anisole, propyl gallate, and C.-tocopherol, DL-tocopherol, C-tocopherol acetate, C.-tocopherol Tocopherol Polyethylene Glycol Succinate (Vitamin E TPGS), L-cysteine ascorbyl palmitate thioglycolic acid, sodium metabisulfite (SMBS), ascorbic acid, sodium formaldehyde sulfoxylate, or hydrophilic antioxidants, including sodium EDTA and thioglycerol. Most typically, the concentration of the anti-oxidant will be between 0.005% and 5% weight/weight of the total composition.

The formulation can include preservative selected from phenol, thimerosal, chlorobutanol, benzyl alcohol, m-cresol, phenoxyethanol, methylparaben and propylparaben typically at a concentration of between 0.001% weight/weight and about 5% weight/weight of the total composition, and is most typically between about 0.003% and about 2.0% weight/weight of the total composition.

The formulation can include acidifying agent selected from citric acid, acetic acid, maleic acid, phosphoric acid, succinic acid, tartaric acid, ascorbic acid, benzene sulfonic acid, oxalic acid, fumaric acid, gluconic acid, malic acid, methane sulfonic acid, p-toluenesulfonic acid, naphthalene sulfonic acid, lacturonic acids, lactic acid, lactobionic acid, edetic acid, gentisic acid, meta phosphoric acid, nitric acid, pentetic acid, glycolic acid. The purpose of using acidifying agents for the present invention is to maintain the acidic pH thereby solubilizing carfilzomib or its pharmaceutically acceptable salts in infusion media. Further, the purpose of using acidifying agents for the present invention is to avoid the precipitation of drug in infusion media.

Certain compounds have been identified as impurities obtained from carfilzomib degradation and have been analysed on stability samples such as [Acid Impurity] (S)-2-((S)-4-methyl-2-((S)-2-(2-morpholinoacetamido)-4-phenyl butanamido)pentanamido)-3 -phenylpropanoic acid; [Diastereomer impurity] (S)-4-methyl-N-((R)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide; [Phenol impurity] 2,3,4,5,6-pentaflurophenol;

[Diol impurity] (S)-N-((S)-1-(((2R,4S)-1,2-dihydroxy-2,6-dimethyl-3-oxoheptan-4-yl)amino)-1-oxo-3-phenylpropan-2-yl)-4-methyl-2-((S)-2-(2-morpholino acetamido)-4-phenylbutanamido)pentanamide; [Chloro impurity] (S)-N-((S)-1-(((2S,4S)-1-chloro-2-hydroxy-2,6-dimethyl-3-oxoheptan-4-yl) amino)-1-oxo-3-phenylpropan-2-yl)-4-methyl-2-((S)-2-(2-morpholinoacetamido)-4-phenylbutanamido)pentanamide; [N-oxide impurity] 4-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)morpholine-4-oxide.

In one embodiment, a room temperature stable formulation which contains no more than total of 6% of impurities formed over the storage period. In one embodiment, a room temperature stable formulation which contains no more than total of 5% of impurities formed over the storage period. In one embodiment, a room temperature stable formulation which contains no more than total of 4% of impurities formed over the storage period. In one embodiment, a room temperature stable formulation which contains no more than total of 3% of impurities formed over the storage period. In one embodiment, a room temperature stable formulation which contains no more than total of 2% of impurities formed over the storage period. In one embodiment, a room temperature stable formulation which contains no more than total of 1% of impurities formed over the storage period. In one embodiment, a room temperature stable formulation which contains no more than total of 0.5% of impurities formed over the storage period. In one embodiment, the storage period of the formulation of invention can be reasonable period of tiqme in which the formulation has sufficient chemical and physical stability. The storage period can be selected such at least about one month, at least about three months, at least about six months, at least about one year, or at least about 2 years.

In one embodiment, the room temperature stable formulation refers to any preparation of carfilzomib having sufficient stability to allow storage at a room temperature, such as between about 15° C. to about 40° C.; preferably between about 20° C. and about 40° C.; more preferably between about 25° C. and about 40° C. ; most preferably at temperature between about 20 and about 25° C. It is to be understood that the stability of the formulation of carfilzomib in the temperature range of the embodiments is always accompanied by additional parameter of 60% humidity. In preferred embodiments of the present invention, stability of room temperature stable formulation may be assessed after storing the formulation of the present invention in a sealed, sterile container at 60% relative humidity at a temperature of 25° C.

In one embodiment the present invention provides room temperature stable carfilzomib formulation which is stable over a period of at least one month when stored at 40° C. and at relative humidity 75%. In one embodiment the present invention provides room temperature stable carfilzomib formulation which is stable over a period of at least three months when stored at 40° C. and at relative humidity 75%. In one embodiment the present invention provides room temperature stable carfilzomib formulation which is stable over a period of at least six months when stored at 40° C. and at relative humidity 75%. In one embodiment the present invention provides room temperature stable carfilzomib formulation which is stable over a period of at least one year when stored at 40° C. and at relative humidity 75%.

In one embodiment the present invention provides room temperature stable carfilzomib formulation which is stable over a period of at least two year when stored at 40° C. and at relative humidity 75%. The stability of the formulation of the present invention is measured by the amount of total impurities formed at the end of stability period. In one embodiment the stability is achieved when impurities formed during assigned stability period is not more than 6%, preferably not more than 5%, preferably not more than 4%, preferably not more than 3%, preferably not more than 2%, preferably not more than 1.5%, preferably not more than 1%, preferably not more than 0.5%.

In one embodiment, after storage for one month at 75% RH and at temperature of 40° C., the carfilzomib compositions can have no more than 6% of total impurity by HPLC method. In one embodiment, after storage for one month at 75% RH and at temperature of 40° C., the carfilzomib compositions can have no more than 5% of total impurity by HPLC method. In one embodiment, after storage for one month at 75% RH and at temperature of 40° C., the carfilzomib compositions can have no more than 4% of total impurity by HPLC. In one embodiment, after storage for one month at 75% RH and at temperature of 40° C., the carfilzomib compositions can have no more than 3% of total impurity by HPLC. In one embodiment, after storage for one month at 75% RH and at temperature of 40° C., the carfilzomib compositions can have no more than 2% of total impurity by HPLC. In one embodiment, after storage for one month at 75% RH and at a temperature of 40° C., the carfilzomib compositions can have no more than 1.5% of total impurity by HPLC. In one embodiment, after storage for one month at 75% RH and at a temperature of 40° C., the carfilzomib compositions can have no more than 1% of total impurity by HPLC. In one embodiment, after storage for 1 month at 75% RH and at a temperature of 40° C., the carfilzomib compositions can have no more than 0.5% of total impurity by HPLC.

In one embodiment, after storage for three months at 75% RH and at temperature of 40° C., the carfilzomib compositions can have no more than 6% of total impurity by HPLC method. In one embodiment, after storage for three months at 75% RH and at temperature of 40° C., the carfilzomib compositions can have no more than 5% of total impurity by HPLC method. In one embodiment, after storage for three months at 75% RH and at temperature of 40° C., the carfilzomib compositions can have no more than 4% of total impurity by HPLC. In one embodiment, after storage for three months at 75% RH and at temperature of 40° C., the carfilzomib compositions can have no more than 3% of total impurity by HPLC. In one embodiment, after storage for three months at 75% RH and at temperature of 40° C., the carfilzomib compositions can have no more than 2% of total impurity by HPLC. In one embodiment, after storage for three months at 75% RH and at a temperature of 40° C., the carfilzomib compositions can have no more than 1.5% of total impurity by HPLC. In one embodiment, after storage for three month at 75% RH and at a temperature of 40° C., the carfilzomib compositions can have no more than 1% of total impurity by HPLC. In one embodiment, after storage for three months at 75% RH and at a temperature of 40° C., the carfilzomib compositions can have no more than 0.5% of total impurity by HPLC.

In one embodiment, after storage for six months at 75% RH and at temperature of 40° C., the carfilzomib compositions can have no more than 6% of total impurity by HPLC method. In one embodiment, after storage for six months at 75% RH and at temperature of 40° C., the carfilzomib compositions can have no more than 5% of total impurity by HPLC method. In one embodiment, after storage for six months at 75% RH and at temperature of 40° C., the carfilzomib compositions can have no more than 4% of total impurity by HPLC. In one embodiment, after storage for six months at 75% RH and at temperature of 40° C., the carfilzomib compositions can have no more than 3% of total impurity by HPLC. In one embodiment, after storage for six months at 75% RH and at temperature of 40° C., the carfilzomib compositions can have no more than 2% of total impurity by HPLC. In one embodiment, after storage for six month at 75% RH and at a temperature of 40° C., the carfilzomib compositions can have no more than 1.5% of total impurity by HPLC. In one embodiment, after storage for six months at 75% RH and at a temperature of 40° C., the carfilzomib compositions can have no more than 1% of total impurity by HPLC. In one embodiment, after storage for six months at 75% RH and at a temperature of 40° C., the carfilzomib compositions can have no more than 0.5% of total impurity by HPLC.

In one embodiment the present invention provides room temperature stable carfilzomib formulation which is stable over a period of at least one month when stored at 25° C. and at relative humidity 60%. In one embodiment the present invention provides room temperature stable carfilzomib formulation which is stable over a period of at least three months when stored at 25° C. and at relative humidity 60%. In one embodiment the present invention provides room temperature stable carfilzomib formulation which is stable over a period of at least six months when stored at 25° C. and at relative humidity 60%. In one embodiment the present invention provides room temperature stable carfilzomib formulation which is stable over a period of at least one year when stored at 25° C. and at relative humidity 60%.

In one embodiment the present invention provides room temperature stable carfilzomib formulation which is stable over a period of at least two year when stored at 25° C. and at relative humidity 60%. Ths stability of the formulation of the present invention is measured by the amount of total impurities formed at the end of stability period. In one embodiment the stability is achieved when impurities formed during assigned stability period is not more than 6%, preferably not more than 5%, preferably not more than 4%, preferably not more than 3%, preferably not more than 2%, preferably not more than 1.5%, preferably not more than 1%, preferably not more than 0.5%.

In one embodiment, after storage for one month at 60% RH and at temperature of 25° C., the carfilzomib compositions can have no more than 6% of total impurity by HPLC method. In one embodiment, after storage for one month at 60% RH and at temperature of 25° C., the carfilzomib compositions can have no more than 5% of total impurity by HPLC method. In one embodiment, after storage for one month at 60% RH and at temperature of 25° C., the carfilzomib compositions can have no more than 4% of total impurity by HPLC. In one embodiment, after storage for one month at 60% RH and at temperature of 25° C., the carfilzomib compositions can have no more than 3% of total impurity by HPLC. In one embodiment, after storage for one month at 60% RH and at temperature of 25° C., the carfilzomib compositions can have no more than 2% of total impurity by HPLC. In one embodiment, after storage for one month at 60% RH and at a temperature of 25° C., the carfilzomib compositions can have no more than 1.5% of total impurity by HPLC. In one embodiment, after storage for one month at 60% RH and at a temperature of 25° C., the carfilzomib compositions can have no more than 1% of total impurity by HPLC. In one embodiment, after storage for 1 month at 60% RH and at a temperature of 25° C., the carfilzomib compositions can have no more than 0.5% of total impurity by HPLC.

In one embodiment, after storage for three months at 60% RH and at temperature of 25° C., the carfilzomib compositions can have no more than 6% of total impurity by HPLC method. In one embodiment, after storage for three months at 60% RH and at temperature of 25° C., the carfilzomib compositions can have no more than 5% of total impurity by HPLC method. In one embodiment, after storage for three months at 60% RH and at temperature of 25° C., the carfilzomib compositions can have no more than 4% of total impurity by HPLC. In one embodiment, after storage for three months at 60% RH and at temperature of 25° C., the carfilzomib compositions can have no more than 3% of total impurity by HPLC. In one embodiment, after storage for three months at 60% RH and at temperature of 25° C., the carfilzomib compositions can have no more than 2% of total impurity by HPLC. In one embodiment, after storage for three months at 60% RH and at a temperature of 25° C., the carfilzomib compositions can have no more than 1.5% of total impurity by HPLC. In one embodiment, after storage for three month at 60% RH and at a temperature of 25° C., the carfilzomib compositions can have no more than 1% of total impurity by HPLC. In one embodiment, after storage for three months at 60% RH and at a temperature of 25° C., the carfilzomib compositions can have no more than 0.5% of total impurity by HPLC.

In one embodiment, after storage for six months at 60% RH and at temperature of 25° C., the carfilzomib compositions can have no more than 6% of total impurity by HPLC method. In one embodiment, after storage for six months at 60% RH and at temperature of 25° C., the carfilzomib compositions can have no more than 5% of total impurity by HPLC method. In one embodiment, after storage for six months at 60% RH and at temperature of 25° C., the carfilzomib compositions can have no more than 4% of total impurity by HPLC. In one embodiment, after storage for six months at 60% RH and at temperature of 25° C., the carfilzomib compositions can have no more than 3% of total impurity by HPLC. In one embodiment, after storage for six months at 60% RH and at temperature of 25° C., the carfilzomib compositions can have no more than 2% of total impurity by HPLC. In one embodiment, after storage for six month at 60% RH and at a temperature of 25° C., the carfilzomib compositions can have no more than 1.5% of total impurity by HPLC. In one embodiment, after storage for six months at 60% RH and at a temperature of 25° C., the carfilzomib compositions can have no more than 1% of total impurity by HPLC. In one embodiment, after storage for six months at 60% RH and at a temperature of 25° C., the carfilzomib compositions can have no more than 0.5% of total impurity by HPLC.

In one embodiment, the present invention provides room temperature stable injectable formulation comprising carfilzomib or its pharmaceutically acceptable salts thereof and one or more solvents.

In one embodiment, the present invention provides room temperature stable injectable formulation comprising carfilzomib or its pharmaceutically acceptable salts thereof; one or more solvent; and optionally one or more pharmaceutically acceptable excipients selected from antioxidant, buffer, preservative and surfactant.

In one embodiment, the present invention provides room temperature stable injectable formulation comprising carfilzomib or its pharmaceutically acceptable salts thereof and one or more solvents; wherein the injectable formulation includes ready to dilute, ready to use and liquid concentrate. In a preferred embodiment, the injectable formulation of the present invention includes room temperature stable ready to dilute solution.

In one embodiment, the present invention provides room temperature stable ready to dilute injectable formulation comprising carfilzomib or its pharmaceutically acceptable salts thereof and one or more solvents.

In one embodiment, the present invention provides room temperature stable ready to dilute injectable formulation comprising carfilzomib or its pharmaceutically acceptable salts thereof; one or more solvents; and optionally one or more pharmaceutically acceptable excipients selected from antioxidant, buffer, preservative and surfactant.

In one embodiment, the present invention provides room temperature stable ready to dilute injectable formulation comprising carfilzomib or its pharmaceutically acceptable salts thereof and one or more solvents; wherein the formulation contains no more than 6% total impurities upon storage of one month.

In one embodiment, the present invention provides room temperature stable ready to dilute injectable formulation comprising carfilzomib or its pharmaceutically acceptable salts thereof and one or more solvents; wherein the formulation contains no more than 5% total impurities upon storage of one month.

In one embodiment, the present invention provides room temperature stable ready to dilute injectable formulation comprising carfilzomib or its pharmaceutically acceptable salts thereof and one or more solvents; wherein the formulation contains no more than 4% total impurities upon storage of one month.

In one embodiment, the present invention provides room temperature stable ready to dilute injectable formulation comprising carfilzomib or its pharmaceutically acceptable salts thereof and one or more solvents; wherein the formulation contains no more than 3% total impurities upon storage of one month.

In one embodiment, the present invention provides room temperature stable ready to dilute injectable formulation comprising carfilzomib or its pharmaceutically acceptable salts thereof and one or more solvents; wherein the formulation contains no more than 2% total impurities upon storage of one month.

In one embodiment, the present invention provides room temperature stable ready to dilute injectable formulation comprising carfilzomib or its pharmaceutically acceptable salts thereof and one or more solvents; wherein the formulation contains no more than 1.5% total impurities upon storage of one month.

In one embodiment, the present invention provides room temperature stable ready to dilute injectable formulation comprising carfilzomib or its pharmaceutically acceptable salts thereof and one or more solvents; wherein the formulation contains no more than 1.0% total impurities upon storage of one month.

In one embodiment, the present invention provides room temperature stable ready to dilute injectable formulation comprising carfilzomib or its pharmaceutically acceptable salts thereof and one or more solvents; wherein the formulation contains no more than 0.5% total impurities upon storage of one month.

In one embodiment, the present invention provides stable ready to dilute injectable formulation comprising carfilzomib or its pharmaceutically acceptable derivatives thereof and one or more solvents; wherein the formulation contains no more than 6% total impurities upon storage of one month when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; wherein the formulation contains no more than 6% of total impurities upon storage of one month when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 5% of total impurities upon storage of one month when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; wherein the formulation contains no more than 4% of total impurities upon storage of one month when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 3% of total impurities upon storage of one month when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 2% of total impurities upon storage of one month when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 1.5% of total impurities upon storage of one month when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 1.0% of total impurities upon storage of one month when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 0.5% of total impurities upon storage of one month when stored at 25° C. and 60% relative humidity.

In one embodiment, the present invention provides room temperature stable ready to dilute injectable formulation comprising carfilzomib or its pharmaceutically acceptable salts thereof and one or more solvents; wherein the formulation contains no more than 6% total impurities upon storage of three months.

In one embodiment, the present invention provides room temperature stable ready to dilute injectable formulation comprising carfilzomib or its pharmaceutically acceptable salts thereof and one or more solvents; wherein the formulation contains no more than 5% total impurities upon storage of three months.

In one embodiment, the present invention provides room temperature stable ready to dilute injectable formulation comprising carfilzomib or its pharmaceutically acceptable salts thereof and one or more solvents; wherein the formulation contains no more than 4% total impurities upon storage of three months.

In one embodiment, the present invention provides room temperature stable ready to dilute injectable formulation comprising carfilzomib or its pharmaceutically acceptable salts thereof and one or more solvents; wherein the formulation contains no more than 3% total impurities upon storage of three months.

In one embodiment, the present invention provides room temperature stable ready to dilute injectable formulation comprising carfilzomib or its pharmaceutically acceptable salts thereof and one or more solvents; wherein the formulation contains no more than 2% total impurities upon storage of three months.

In one embodiment, the present invention provides room temperature stable ready to dilute injectable formulation comprising carfilzomib or its pharmaceutically acceptable salts thereof and one or more solvents; wherein the formulation contains no more than 1.5% total impurities upon storage of three months.

In one embodiment, the present invention provides room temperature stable ready to dilute injectable formulation comprising carfilzomib or its pharmaceutically acceptable salts thereof and one or more solvents; wherein the formulation contains no more than 1.0% total impurities upon storage of three months.

In one embodiment, the present invention provides room temperature stable ready to dilute injectable formulation comprising carfilzomib or its pharmaceutically acceptable salts thereof and one or more solvents; wherein the formulation contains no more than 0.5% total impurities upon storage of three months.

In one embodiment, the present invention provides stable ready to dilute injectable formulation comprising carfilzomib or its pharmaceutically acceptable derivatives thereof and one or more solvents; wherein the formulation contains no more than 6% total impurities upon storage of three months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; wherein the formulation contains no more than 6% of total impurities upon storage of three months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; wherein the formulation contains no more than 5% of total impurities upon storage of three months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 4% of total impurities upon storage of three months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 3% of total impurities upon storage of three months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 2% of total impurities upon storage of three months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 1.5% of total impurities upon storage of three months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 1.0% of total impurities upon storage of three months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 0.5% of total impurities upon storage of three months when stored at 25° C. and 60% relative humidity.

In one embodiment, the present invention provides room temperature stable ready to dilute injectable formulation comprising carfilzomib or its pharmaceutically acceptable salts thereof and one or more solvents; wherein the formulation contains no more than 6% total impurities upon storage of six months.

In one embodiment, the present invention provides room temperature stable ready to dilute injectable formulation comprising carfilzomib or its pharmaceutically acceptable salts thereof and one or more solvents; wherein the formulation contains no more than 5% total impurities upon storage of six months.

In one embodiment, the present invention provides room temperature stable ready to dilute injectable formulation comprising carfilzomib or its pharmaceutically acceptable salts thereof and one or more solvents; wherein the formulation contains no more than 4% total impurities upon storage of six months.

In one embodiment, the present invention provides room temperature stable ready to dilute injectable formulation comprising carfilzomib or its pharmaceutically acceptable salts thereof and one or more solvents; wherein the formulation contains no more than 3% total impurities upon storage of six months.

In one embodiment, the present invention provides room temperature stable ready to dilute injectable formulation comprising carfilzomib or its pharmaceutically acceptable salts thereof and one or more solvents; wherein the formulation contains no more than 2% total impurities upon storage of six months.

In one embodiment, the present invention provides room temperature stable ready to dilute injectable formulation comprising carfilzomib or its pharmaceutically acceptable salts thereof and one or more solvents; wherein the formulation contains no more than 1.5% total impurities upon storage of six months.

In one embodiment, the present invention provides room temperature stable ready to dilute injectable formulation comprising carfilzomib or its pharmaceutically acceptable salts thereof and one or more solvents; wherein the formulation contains no more than 1.0% total impurities upon storage of six months.

In one embodiment, the present invention provides room temperature stable ready to dilute injectable formulation comprising carfilzomib or its pharmaceutically acceptable salts thereof and one or more solvents; wherein the formulation contains no more than 0.5% total impurities upon storage of six months.

In one embodiment, the present invention provides stable ready to dilute injectable formulation comprising carfilzomib or its pharmaceutically acceptable derivatives thereof and one or more solvents; wherein the formulation contains no more than 6% total impurities upon storage of six months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 6% of total impurities upon storage of six months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 5% of total impurities upon storage of six months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 4% of total impurities upon storage of six months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 3% of total impurities upon storage of six months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 2% of total impurities upon storage of six months when stored at 25° C. and 60% relative humidity.

In one embodiment, the present invention provides stable ready to dilute injectable formulation comprising carfilzomib aor its pharmaceutically acceptable salts thereof; one or more solvent; and optionally one or more pharmaceutically acceptable excipients selected from antioxidant, buffer, preservative and surfactant; where in the formulation contains no more than 1.5% of total impurities upon storage of six months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 1.0% of total impurities upon storage of six months when stored at 25° C. and 60% relative humidity.

In one embodiment, the present invention provides stable ready to dilute injectable formulation comprising carfilzomib or its pharmaceutically acceptable salts thereof; one or more solvent; and optionally one or more pharmaceutically acceptable excipients selected from antioxidant, buffer, preservative and surfactant; where in the formulation contains no more than 0.5% of total impurities upon storage of six months when stored at 25 oC and 60% relative humidity.

It is always desirable and beneficial to have stable room temperature injectable formulation which has commercial and handling benefits over formulation stored as stringent conditions such as 2-8° C. However, it is very challenging to obtain injectable formulation which is stable at room temperature having less impurity or impurity level within acceptable limit by drug approval authority.

During stability study period, surprisingly it has been found that the ready to dilute injectable solution without acidifying agent has a significantly better impurity profile than ready to dilute injectable solution with acidifying agent. Thus, acidifying agent impart negative impact on the stability of formulation at room temperature and generates more impurities during stability period than the formulation without acidifying agent. The better impurity profile according to present invention includes difference in the total impurities obtained during the stability period upon analysis of formulation sample on HPLC.

In one embodiment, the present invention provides room temperature stabilized injectable formulation comprising carfilzomib and its pharmaceutically acceptable salts which does not contain any acidifying agent.

In one embodiment, the present invention provides room temperature stabilized injectable formulation comprising Carfilzomib and its pharmaceutically acceptable salts which does not contain any acidifying agent during the stability period of the formulation.

In one embodiment, the present invention provides room temperature stable ready to dilute injectable formulation comprising Carfilzomib and its pharmaceutically acceptable salts; one or more solvent; and optionally one or more excipients selected from anti-oxidant, buffer and surfactant; and does not contain any acidifying agent during the stability period of the formulation.

In one embodiment, the present invention provides room temperature stable ready to dilute injectable formulation comprising carfilzomib and its pharmaceutically acceptable salts; one or more solvent; and optionally one or more excipients selected from anti-oxidant, buffer and surfactant; and does not contain any acidifying agent during the stability period of the formulation.

In one embodiment of present invention is directed to delivery of room temperature stable carfilzomib injectable ready to dilute formulation, which once diluted to appropriate injection (especially infusion, most particularly IV infusion) concentrations, it may be administered in appropriate amounts for treating carfilzomib responsive conditions known in the art.

In one embodiment of the present invention is provided a method for treating patients with relapsed or refractory multiple myeloma by administering room temperature stable ready to dilute or ready-to-use parenteral formulation of carfilzomib either alone or in combination with dexamethasone or lenalidomide plus dexamethasone.

In one embodiment of the present invention is provided a method for treating patients with relapsed or refractory multiple myeloma comprises administering room temperature stable ready to dilute injectable formulation of carfilzomib or its pharmaceutically acceptable salts thereof; and one or more solvents; wherein the formulation contains no more than 6% total impurities upon storage period of formulation. In one embodiment the method of treating patients with relapsed or refractory multiple myeloma includes administering the formulation of carfilzomib or its pharmaceutically acceptable salts that contains no more than 5% total impurities upon storage period of formulation. In one embodiment the method of treating patients with relapsed or refractory multiple myeloma includes administering the formulation of carfilzomib or its pharmaceutically acceptable salts that contains no more than 4% total impurities upon storage period of formulation. In one embodiment the method of treating patients with relapsed or refractory multiple myeloma includes administering the formulation of carfilzomib or its pharmaceutically acceptable salts that contains no more than 3% total impurities upon storage period of formulation. In one embodiment the method of treating patients with relapsed or refractory multiple myeloma includes administering the formulation of carfilzomib or its pharmaceutically acceptable salts that contains no more than 2% total impurities upon storage period of formulation. In one embodiment the method of treating patients with relapsed or refractory multiple myeloma includes administering the formulation of carfilzomib or its pharmaceutically acceptable salts that contains no more than 1.5% total impurities upon storage period of formulation. In one embodiment the method of treating patients with relapsed or refractory multiple myeloma includes administering the formulation of carfilzomib or its pharmaceutically acceptable salts that contains no more than 1% total impurities upon storage period of formulation. In one embodiment the method of treating patients with relapsed or refractory multiple myeloma includes administering the formulation of carfilzomib or its pharmaceutically acceptable salts that contains no more than 0.5% total impurities upon storage period of formulation.

In one embodiment of the present invention is provided the method for treating patients with relapsed or refractory multiple myeloma comprises administering room temperature stable ready to dilute injectable formulation of carfilzomib or its pharmaceutically acceptable salts thereof, wherein the ready to dilute injectable formulation is stable for one month when stored at 25° C. and 60% relative humidity. Preferably In one embodiment of the present invention is provided the method for treating patients with relapsed or refractory multiple myeloma comprises administering room temperature stable ready to dilute injectable formulation of carfilzomib or its pharmaceutically acceptable salts thereof, wherein the ready to dilute injectable formulation is stable for three months when stored at 25° C. and 60% relative humidity. More preferably, in one embodiment of the present invention is provided the method for treating patients with relapsed or refractory multiple myeloma comprises administering room temperature stable ready to dilute injectable formulation of carfilzomib or its pharmaceutically acceptable salts thereof, wherein the ready to dilute injectable formulation is stable for six months when stored at 25° C. and 60% relative humidity.

In one embodiment of the present invention is provided a method for treating patients with relapsed or refractory multiple myeloma which includes method of administering reconstituted ready to dilute solution comprising mixing of component 1 and component 2 before diluting with infusion media; wherein the component 1 comprises room temperature stable ready to dilute parenteral formulation of carfilzomib or its pharmaceutically acceptable salts and component 2 is acidifying agent.

In one embodiment of the present invention component 2 comprises the acidifying agent which can be selected from citric acid, malic acid, phosphoric acid, orthophosphoric acid (OPA), fumaric acid or mixtures thereof. In one embodiment the ratio of acidifying agent used in comparison to carfilzomib or its pharmaceutically acceptable salt is from about 1:50 to about 1:1 on the basis of w/w. In one embodiment the ratio of acidifying agent used in comparison to carfilzomib or its pharmaceutically acceptable salt is from about 1:40 to about 1:1 on the basis of w/w. In one embodiment the ratio of acidifying agent used in comparison to carfilzomib or its pharmaceutically acceptable salt is from about 1:30 to about 1:1 on the basis of w/w. In one embodiment the ratio of acidifying agent used in comparison to carfilzomib or its pharmaceutically acceptable salt is from about 1:20 to about 1:1 on the basis of w/w. In one embodiment the ratio of acidifying agent used in comparison to carfilzomib or its pharmaceutically acceptable salt is from about 1:10 to about 1:1 on the basis of w/w.

The addition of component 2 into component 1 makes Carfilzomib or its pharmaceutical acceptable salts susceptible to degradation at room temperature in ready to dilute parenteral solution. In one embodiment, mixture comprising component 1 and component 2 should not be used after 4 hours of mixing when the mixture is stored at room temperature. In a preferred embodiment, the mixture of component 1 and component 2 should not be used after 5 hours of mixing when the mixture is stored at room temperature. In a preferred embodiment, the mixture of component 1 and component 2 should not be used after 6 hours of mixing when the mixture is stored at room temperature.

In a preferred embodiment, the mixture of component 1 and component 2 should not be used for administration into infusion media after 24 hours of mixing when the mixture is stored at 2-8° C.

In one embodiment of the present invention is provided a method for treating patients with relapsed or refractory multiple myeloma which includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent; and wherein the mixture of component 1 and component 2 is not used for administration after 4 hours of mixing when the mixture is stored at room temperature.

In one embodiment of the present invention is provided a method for treating patients with relapsed or refractory multiple myeloma includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent and wherein the mixture of component 1 and component 2 is not used for administration after 5 hours of mixing when the mixture is stored at room temperature.

In one embodiment of the present invention is provided a method for treating patients with relapsed or refractory multiple myeloma includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent; and wherein the mixture of component 1 and component 2 is not used for administration after 6 hours of mixing when the mixture is stored at room temperature.

In one embodiment of the present invention is provided a method for treating patients with relapsed or refractory multiple myeloma which includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent and wherein reconstituted ready to dilute composition is having total impurity not more than 2% upto 4 hours after mixing when the mixture is stored at room temperature.

In one embodiment of the present invention is provided a method for treating patients with relapsed or refractory multiple myeloma which includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent and wherein reconstituted ready to dilute composition is having total impurity not more than 1% upto 4 hours after mixing when the mixture is stored at room temperature.

In one embodiment of the present invention is provided a method for treating patients with relapsed or refractory multiple myeloma which includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent and wherein reconstituted ready to dilute composition is having total impurity not more than 2% upto 5 hours after mixing when the mixture is stored at room temperature.

In one embodiment of the present invention is provided a method for treating patients with relapsed or refractory multiple myeloma which includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent and wherein reconstituted ready to dilute composition is having total impurity not more than 1% upto 5 hours after mixing when the mixture is stored at room temperature.

In one embodiment of the present invention is provided a method for treating patients with relapsed or refractory multiple myeloma which includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent and wherein reconstituted ready to dilute composition is having total impurity not more than 2% upto 6 hours after mixing when the mixture is stored at room temperature.

In one embodiment of the present invention is provided a method for treating patients with relapsed or refractory multiple myeloma which includes method of administering reconstituted ready to dilute solution comprising mixing of component 1 and component 2 to form reconstituted ready to dilute solution followed by diluting reconstituted ready to dilute solution with infusion media; wherein the component 1 is room temperature stable ready to dilute parenteral formulation of carfilzomib or its pharmaceutically acceptable salts and component 2 is acidifying agent and wherein reconstituted ready to dilute composition is having total impurity not more than 1% up to 6 hours after mixing when the mixture is stored at room temperature.

In one embodiment of the present invention is provided a method for treating patients with relapsed or refractory multiple myeloma includes method of administering reconstituted ready to dilute solution comprising mixing of component 1 and component 2 to form reconstituted ready to dilute solution followed by diluting reconstituted ready to dilute solution with infusion media; wherein the component 1 comprises administering room temperature stable ready to dilute parenteral formulation of carfilzomib or its pharmaceutically acceptable salts and component 2 is acidifying agent; and wherein the mixture of component 1 and component 2 is not used for administration after 24 hours of mixing when the mixture is stored at 2-8 oC.

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of carfilzomib or its pharmaceutically acceptable salts and component 2 is acidifying agent.

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of carfilzomib or its pharmaceutically acceptable salts and contains no more than 6% of total impurities upon storage of six months when stored at 25° C. and 60% relative humidity; and component 2 is acidifying agent;

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of carfilzomib or its pharmaceutically acceptable salts and contains no more than 5% of total impurities upon storage of six months when stored at 25° C. and 60% relative humidity; and component 2 is acidifying agent.

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of carfilzomib or its pharmaceutically acceptable salts and contains no more than 4% of total impurities upon storage of six months when stored at 25° C. and 60% relative humidity; and component 2 is acidifying agent.

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of Carfilzomib or its pharmaceutically acceptable salts and no more than 3% of total impurities upon storage of six months when stored at 25° C. and 60% relative humidity; and component 2 is acidifying agent.

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of carfilzomib or its pharmaceutically acceptable salts and contains no more than 2% of total impurities upon storage of six months when stored at 25° C. and 60% relative humidity; and component 2 is acidifying agent.

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of carfilzomib or its pharmaceutically acceptable salts and contains no more than 1.5% of total impurities upon storage of six months when stored at 25° C. and 60% relative humidity; and component 2 is acidifying agent.

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of carfilzomib or its pharmaceutically acceptable salts and contains no more than 1% of total impurities upon storage of six months when stored at 25° C. and 60% relative humidity; and component 2 is acidifying agent.

In one embodiment of the present invention is provided a method of administering reconstituted ready to dilute solution comprising mixing of component 1 and component 2 to form reconstituted ready to dilute solution followed by diluting reconstituted ready to dilute solution with infusion media; wherein the component 1 comprises room temperature stable ready to dilute parenteral formulation of carfilzomib or its pharmaceutically acceptable salts and contains no more than 0.5% of total impurities upon storage of six months when stored at 25° C. and 60% relative humidity; and component 2 is acidifying agent.

The following examples are given for the purpose of illustrating the present invention and should not be considered as limiting the scope of the invention.

### EXAMPLE 1

### Carfilzomib Formulation Preparation

| Ingredients (Batch number) | APPL-006/01/068 | |
|---|---|---|
| Carfilzomib | 30 | mg |
| Ortho phosphoric acid | 10 | μl |
| Dimethyl acetamide | 500 | μl |
| Dehydrated alcohol (ethanol) | 500 | μl |

### Procedure:

1. Partial amount of the Dimethyl acetamide was taken into vessel and weighted amount of carfilzomib was added to it and mixed under continuous stirring under nitrogen purging and sodium vapor lamp.
2. Dehydrated alcohol was added to above solvent system and mixed until the clear solution was obtained.
3. Weighed amount of ortho phosphoric acid was added under continuous stirring and mixed well.
4. Final volume make up was done using dimethyl acetamide and mixed well to uniform solution.
5. Product was filtered with 0.22 μ filter and filled into vial.

### EXAMPLE 2

### Carfilzomib Formulation Preparation

| Ingredients (Batch number) | APPL-006/01/033 | |
|---|---|---|
| Carfilzomib | 10 | mg |
| Propylene Glycol | 425 | μl |
| Polysorbate 80 | 300 | μl |
| Alpha-Tocopherol | 0.2 | mg |
| Lactic acid | qs to pH 3-4 | |
| DMA | 200 | μl |

### Procedure:

1. Propylene glycol, polysorbate 80 were mixed at 2-8° C. for 10 minutes.
2. Alpha tocopherol was added to the above mixture.
3. pH of the solution was adjusted to 3-4 (3.5) using lactic acid.
4. Carfilzomib and dimethyl acetamide mixture were added to the step 3, and mixed until the clear solution.
5. Nitrogen purging and light protection was done throughout the batch preparation at 2-8° C.
6. The product was filtered with 0.22 μ and filled in vial and sealed.

### EXAMPLE 2A

### Carfilzomib Formulation Preparation

Examples 2A was prepared in a similar way as shown in example 2

| Ingredients (Batch number) | APPL-006/01/035 | |
|---|---|---|
| Carfilzomib | 10 | mg |
| Propylene Glycol | Qs to mL | |
| Polysorbate 80 | 300 | μl |
| Alpha-Tocopherol | 0.2 | mg |
| Lactic acid | qs to pH 3-4 | |
| Dimethyl acetamide | 200 | μl |
| Water for injection | 30 | mg |

### EXAMPLE 2B

### Carfilzomib Formulation Preparation

Examples 2B was prepared in a similar way as shown in example 2

| Ingredients (Batch number) | APPL-006/01/036 | |
|---|---|---|
| Carfilzomib | 10 | mg |
| Propylene Glycol | Qs to mL | |
| Polysorbate 80 | 300 | μl |
| Lactic acid | qs to pH 3-4 | |
| Dimethyl acetamide | 200 | μl |
| Butylated Hydroxy Toluene | 0.02 | mg |

### EXAMPLE 2C

### Carfilzomib Formulation Preparation

Examples 2C was prepared in a similar way as shown in example 2

| Ingredients (Batch number) | APPL-006/01/037 | |
|---|---|---|
| Carfilzomib | 10 | mg |
| Propylene Glycol | Qs to mL | |
| Polysorbate 80 | 300 | μl |
| Lactic acid | qs to pH 3-4 | |
| Dimethyl acetamide | 200 | μl |
| Water for injection | 30 | mg |
| Butylated Hydroxy Toluene | 0.02 | mg |

### EXAMPLE 3

### Carfilzomib Formulation Preparation

| Ingredients (Batch number) | APPL-006/01/069 | |
|---|---|---|
| Carfilzomib | 30 | mg |
| Dimethyl acetamide | 500 | μl |
| Dehydrated alcohol (ethanol) | 500 | μl |

### Procedure:

1. Dimethyl acetamide and dehydrated alcohol were mixed uniformly at room temperature under nitrogen purging and sodium vapor lamp.
2. Carfilzomib was added to above solvent system and mixed until the complete solubilization.
3. Product was filtered with 0.22 μ filter and filled into vial.

### EXAMPLE 4

### Carfilzomib Formulation Preparation

Formulation T1 to T4 were prepared similar to the procedure described in example 3.

| Ingredients | T1 | | T2 | | T3 | | T4 | |
|---|---|---|---|---|---|---|---|---|
| Carfilzomib | 10 | mg | 30 | mg | 60 | mg | 100 | mg |

| | | | | | | |
|---|---|---|---|---|---|---|
| Dimethyl acetamide | — | — | 1000 | μL | 1000 | μL |

| | | | | | | |
|---|---|---|---|---|---|---|
| Dehydrated alcohol | 1000 | μL | 1000 | μL | — | — |
| (ethanol) | | | | | | |

### EXAMPLE 5

### Carfilzomib Formulation Preparation

| Ingredients (Batch number) | APPL-006/01/062 | |
|---|---|---|
| Carfilzomib | 10 | mg |
| Propylene Glycol | 440 | μl |
| Polysorbate 80 | 300 | μl |
| Alpha-Tocopherol | 0.2 | mg |
| Dimethyl acetamide | 250 | μl |
| Water for injection | 30 | μl |

### Procedure:

1. Dimethyl acetamide was taken into suitable vessel and temp was maintained at 2-8° C.
2. Alpha tocopherol was added to the step 1.
3. Carfilzomib was added to the DMA and tocopherol mixture of step 2 and mixed well until the complete solubilization of the it.
4. Water for injection and polysorbate 80 were added to the above step 3 and mixed well until uniform solution.
5. Volume make up was done using the propylene glycol and mixed until uniform solution.
6. Complete process was done under the nitrogen purging and sodium vapour lamp.
7. Product was filtered with 0.22 μ filter and filled into vial.

### EXAMPLE 5A

Examples 5A was prepared in a similar way as shown in example 5

| Ingredients (Batch number) | APPL-006/01/058 | |
|---|---|---|
| Carfilzomib | 30 | mg |
| Polysorbate 80 | 300 | μl |
| Alpha-Tocopherol | 0.2 | mg |
| Lactic Acid | Qs to pH 3-4 | |
| Dimethyl acetamide | 250 | μl |
| Water for injection | 30 | mg |

| | |
|---|---|
| Super refined PG | QS to 1 ml |

### EXAMPLE 5B

Examples 5B was prepared in a similar way as shown in example 5

| Ingredients (Batch number) | APPL-006/01/059 | |
|---|---|---|
| Carfilzomib | 10 | mg |
| Polysorbate 80 | 300 | μl |
| Alpha-Tocopherol | 0.2 | mg |
| HCL | Qs to pH 3-4 | |
| DMA | 250 | μl |
| Water for injection | 30 | mg |

| | |
|---|---|
| Super refined PG | QS to 1 ml |

TABLE 1
Stability of prepared formulation in example 5A and 5B.

| | | Batch No. | | | | | | |
|---|---|---|---|---|---|---|---|---|
| | | APPL-006/01/058 | | | | APPL-006/01/059 | | |
| Test | Time | Initial | 2-8° C. | RT | 2-8° C. | Initial | 2-8 | RT |
| parameters* | points | | 3 Days | 7 Days | 7 Days | | 3 Days | 7 Days |
| Description | Clear | Clear | Clear | Clear | Clear | Clear | Clear | Clear |
| | colorless | Solution | Solution | Solution | Solution | solution | solution | solution |
| | solution | | | | | | | |
| Related | | | | | | | | |
| Substances | | | | | | | | |
| Acid | NMT | ND | 0.02 | 0.22 | 0.01 | ND | ND | 0.05 |
| Impurity | 0.2% | | | | | | | |
| Phenol | NMT | ND | ND | ND | ND | ND | ND | ND |
| Impurity | 0.2% | | | | | | | |
| Chloro | NMT | ND | ND | ND | ND | ND | 0.09 | ND |
| Impurity | 0.5% | | | | | | | |
| Diol | NMT | ND | 0.02 | 0.18 | 0.02 | 0.01 | 0.01 | 0.06 |
| Impurity | 0.5% | | | | | | | |
| Diastereomer | NMT | ND | ND | ND | ND | ND | ND | ND |
| Impurity | 0.2% | | | | | | | |
| N-Oxide | NMT | 0.02 | 0.02 | 0.11 | 0.02 | 0.02 | 0.02 | 0.02 |
| Impurity | 1.0% | | | | | | | |
| Any other | NMT | 0.06 | 0.06 | 0.36 | 0.02 | 0.06 | 0.06 | 0.39 |
| individual | 0.2% | | | | | | | |
| impurity | | | | | | | | |
| Total | NMT | 0.08 | 0.22 | 2.18 | 0.23 | 0.09 | 0.32 | 4.41 |
| Impurities | 3.0% | | | | | | | |

### EXAMPLE 6

### Carfilzomib Formulation Preparation

Formulation of this example was prepared similar to the procedure described in example 5.

| Ingredients (Batch number) | APPL-006/01/070 | |
|---|---|---|
| Carfilzomib | 15 | mg |
| Propylene Glycol | 220 | μl |
| Polysorbate 80 | 210 | μl |
| Alpha-Tocopherol | 0.2 | mg |
| Dimethyl acetamide | 275 | μl |
| Dehydrated alcohol (ethanol) | 275 | μl |

### EXAMPLE 6A

### Carfilzomib Formulation Preparation

| Ingredients (Batch number) | APPL-006/01/078 | |
|---|---|---|
| Carfilzomib | 30 | mg |
| Alpha-Tocopherol | 0.5 | mg |

| | |
|---|---|
| Dehydrated alcohol (ethanol) | QS to 1 ml |

1. Sufficient quantity of ethanol was taken in a manufacturing vessel.
2. Weighed quantity of alpha-tocopherol was added under continuous stirring until uniform solution obtained. The description was recorded
3. Weighed quantity of Carfilzomib was added under continuous stirring until uniform solution obtained. The description was recorded.
4. Final volume was made-up by ethanol and stirred until uniform solution obtained.
5. The bulk was filtered through 0.22 μ PTFE filter and filled into vial

### EXAMPLE 6B

### Carfilzomib Formulation Preparation

| Ingredients (Batch number) | APPL-006/01/087 | |
|---|---|---|
| Carfilzomib | 30 | mg |
| Alpha-Tocopherol | 0.5 | mg |
| Dimethyl acetamide | 200 | ul |

| | |
|---|---|
| Dehydrated alcohol (ethanol) | QS to 1 mL |

1. Sufficient quantity of N,N-dimethyl acetamide was taken in a manufacturing vessel.
2. Weighed quantity of alpha-tocopherol was added under continuous stirring until uniform solution obtained. The description was recorded
3. Weighed quantity of Carfilzomib was added under continuous stirring until uniform solution obtained. The description was recorded.
4. Final volume was made-up by ethanol and stirred until uniform solution obtained.
5. The bulk was filtered through 0.22 μ PTFE filter and filled into vial.

### EXAMPLE 7

TABLE 2
Stability comparison of prepared formulation of example 1, 2, 3 and 5

| | | Batch No. | | | | | | | |
|---|---|---|---|---|---|---|---|---|---|
| | | APPL- | APPL- | APPL- | APPL- | APPL- | APPL- | APPL- | APPL- |
| | | 006/01/ | 006/01/ | 006/01/ | 006/01/ | 006/01/ | 006/01/ | 006/01/ | 006/01/ |
| | | 033 | 033 | 062 | 062 | 068 | 068 | 069 | 069 |
| Test | Time | RT | RT | RT | RT | RT | RT | RT | RT |
| parameters* | points | initial | After | initial 0 | After | initial | After | intial 0 | After |
| | | 0 days | 21 | days | 35 | 0 days | 24 | days | 24 |
| | | | Days | | Days | | Days | | Days |
| Description | Clear | Clear | Clear | Clear | Clear | Clear | Clear | Clear | Clear |
| | color | Solution | Solution | Solution | Solution | solution | solution | solution | solution |
| | less | | | | | | | | |
| | solution | | | | | | | | |
| Related | | | | | | | | | |
| Substances | | | | | | | | | |
| Acid | NMT | ND | 0.12 | ND | ND | ND | ND | ND | ND |
| Impurity | 0.2% | | | | | | | | |
| Phenol | NMT | ND | ND | ND | ND | ND | ND | ND | ND |
| Impurity | 0.2% | | | | | | | | |
| Chloro | NMT | ND | ND | ND | ND | ND | 0.09 | ND | ND |
| Impurity | 0.5% | | | | | | | | |
| Diol | NMT | ND | 0.16 | ND | 0.15 | 0.01 | 1.9 | 0.01 | ND |
| Impurity | 0.5% | | | | | | | | |
| Diastereomer | NMT | ND | ND | ND | ND | ND | ND | ND | 0.01 |
| Impurity | 0.2% | | | | | | | | |
| N-Oxide | NMT | ND | 1.23 | 0.02 | 0.03 | 0.02 | 0.88 | 0.01 | 0.01 |
| Impurity | 1.0% | | | | | | | | |
| Any | NMT | ND | 0.15 | 0.06 | 0.13 | 0.07 | 2.18 | 0.06 | 0.06 |
| other | 0.2% | | | | | | | | |
| individual | | | | | | | | | |
| impurity | | | | | | | | | |
| Total | NMT | ND | 2.5 | 0.08 | 0.75 | 0.16 | 7.31 | 0.13 | 0.19 |
| Impurities | 3.0% | | | | | | | | |

*RT mean room (Stability test parameter: temperature 25° C. and 60% relative humidity),
*ND means not detected,
*NMT means not more than

### EXAMPLE 8

### Preparation and Stability Data of Injectable Composition Having Acidifying Agent

| Ingredients | Qty/ml | |
|---|---|---|
| Carfilzomib | 30 | mg |
| Alphatocopherol | 0.5 | mg |
| o-phosphoric acid | 15 | uL |

| | |
|---|---|
| Dehydrated ethanol | q.s. to 1 ml |

### Procedure:

1. 80% amount of required Dehydrated alcohol was taken in clean vessel.
2. Required quantity of carfilzomib was added in step 1 solvent at 2-8° C. and dissolved it with mechanical stirrer till clear solution observed.
3. Added required quantity of alphatocopherol with stirring in step 2 at 2-8° C. till clear solution observed.
4. Added required quantity of o-phosphoric acid with stirring in step 3 at 2-8° C. till clear solution observed.
5. Made up the final batch size with required amount of dimethyl acetamide and mixed it well at 2-8° C.
6. Filtered the bulk solution using 0.2-micron PTFE filter.
7. Filled in amber colour vial, followed by nitrogen blanketing and sealed it properly with rubber stopper.
8. The vials were kept on thermal stability at 25° C. and 60% RH and 2-8° C.

TABLE 3
Stability data of injectable composition having acidifying agent

| | storage condition | | | |
|---|---|---|---|---|
| | | 25° C./60% RH | | 2-8° C. |
| | sample condition | | | |
| | Initial | 15 Days | 1 M | 1 M |
| Description | Complies | Complies | Complies | Complies |
| Assay of Carfilzomib | 99.9% | 94.0% | 89.0% | 98.7% |
| Related substances | | | | |
| Acid impurity | ND | 0.16% | 0.45% | 0.04% |
| Phenol impurity | ND | ND | ND | ND |
| Chloro impurity | 0.01% | ND | 1.43% | 0.29% |
| Diol Impurity | ND | 0.13% | 0.00 | 0.03% |
| Diastereomer Impurty | ND | ND | ND | ND |
| N-Oxide Impurity | 0.02% | 0.20% | 0.51% | 0.05% |
| Any other individual | Not Detected | 1.02 | 1.34% | 0.43% |
| impurities | | | | |
| Total Impurities | 0.06% | 5.37% | 8.59% | 1.34% |

### EXAMPLE 9

### Preparation and Stability Data of Injectable Composition Before and After Mixing of Acidifying Agent

| Ingredients | Qty/ml | |
|---|---|---|
| Carfilzomib | 30 | mg |
| Dimethyl acetamide | 500 | μL |
| Dehydrated alcohol (ethanol) | 500 | μL |

Procedure:
1. 80% amount of required dimethyl acetamide was taken in clean vessel.
2. Required quantity of carfilzomib was added in step 1 at 2-8° C. and dissolved it with mechanical stirrer till clear solution observed.
3. Added required quantity of dehydrated Ethanol with stirring in step 2 at 2-8° C. till clear solution observed.
4. Made up the final batch size with required amount of dimethyl acetamide and mixed it well at 2-8° C.
5. Filtered the bulk solution using 0.2-micron PTFE filter.
6. Filled in amber colour vial, followed by nitrogen blanketing and sealed it properly with rubber stopper.
7. The vials were kept on thermal stability at 25° C. /60% RH and 2-8° C.

TABLE 4
Stability data of injectable composition before mixing of acidifying agent

| | | 25° C./60 | 25° C./60 | 25° C./60 | | | |
|---|---|---|---|---|---|---|---|
| | | RH | RH | RH | 2-8° C. | 2-8° C. | 2-8° C. |
| | Initial | 1M | 2M | 3M | 1M | 2M | 3M |
| Description | Clear | Clear | Clear | Clear | Clear | Clear | Clear |
| | solution | solution | solution | solution | solution | solution | solution |
| pH | 8.4 | NP | NP | NP | NP | NP | NP |
| Assay of | 100.1% | 100.5% | 101.10% | 99.2% | 100.1% | 101.40% | 103.1% |
| Carfilzomib | | | | | | | |
| Related | | | | | | | |
| Substances | | | | | | | |
| Diol | ND | 0.02% | 0.03% | 0.04% | 0.01% | 0.01% | 0.02% |
| Impurity | | | | | | | |
| N-Oxide | 0.01% | 0.02% | 0.03% | 0.04% | 0.01% | 0.02% | 0.02% |
| Impurity | | | | | | | |
| Any other | ND | 0.09% | 0.1% | 0.13% | 0.05% | 0.05% | 0.05% |
| individual | | | | | | | |
| impurity | | | | | | | |
| Total | 0.10% | 0.30% | 0.49% | 0.46% | 0.18% | 0.22% | 0.21% |
| Impurities | | | | | | | |

*NP = Not performed

TABLE 5
Procedure for mixing acidifying agent
to ready to dilute composition:

| Product Vial | Diluent vial | | | Final |
|---|---|---|---|---|
| (Vial 1) | (Vial 2) | Vial 1 | Vial 2 | Concentration |
| 30 mg/mL | 90 microliter | 1 ml | 0.5 ml | 20 mg/mL |
| product | OPA up to 1 mL | | | Carfilzomib |
| Example 2 | water for | | | |
| | injection, USP | | | |

1. Aseptically mixed the vial 1 and vial 2 as per the table 3 mentioned above.
2. Gently swirled and/or inverted the vial slowly for about 1 minute.
3. Visually inspected for particulate matter and discoloration prior mixing. The diluted product should be a clear, colorless solution and should not be mixed if any discoloration or particulate matter is observed.
4. The mixed product solution in step 3 was diluted with sterile water for injection at the concentration of 0.5 mg/ml and 1.5 mg/ml and perform the solution stability at the 25° C. and 2-8° C.

TABLE 6
Stability data of injectable composition after mixing of acidifying agent
at concentration of 0.5 mg/ml and 1.5 mg/ml

| | 0.5 mg/ml | | | 1.5 mg/ml | | |
|---|---|---|---|---|---|---|
| Test parameters | Initial | 25° C./4 Hr | 2-8° C./24 Hr | Initial | 25° C./4 Hr | 2-8° C./24 Hr |
| Description | Complies | Complies | Complies | Complies | Complies | Complies |
| pH | 2.21 | 2.48 | 2.40 | 2.10 | 2.27 | 2.45 |
| Osmolarity | 259 | 259 | 258 | 795 | 776 | 776 |
| (msOm/kg) | | | | | | |
| Light | 98.929 | 99.449 | 99.541 | 98.944 | 98.369 | 98.926 |
| Transmittance % | | | | | | |
| Absorbance | 0.007 | 0.006 | 0.007 | 0.012 | 0.013 | 0.014 |
| Carfilzomib | 97.1% | 98.4% | 98.6% | 99.40% | 99.7% | 98.9% |
| Assay | | | | | | |
| Diol Impurity | 0.12% | 0.16% | 0.18% | 0.16% | 0.16% | 0.21% |
| Any other | 0.06% | 0.09% | 0.13% | 0.09% | 0.10% | 0.13% |
| individual | | | | | | |
| impurities | | | | | | |
| Total Impurities | 0.41% | 0.42% | 0.56% | 0.44% | 0.51% | 0.70% |

### EXAMPLE 10

### Preparation and Stability Data of Injectable Composition Before and After Mixing of Acidifying Agent

| Ingredients | Qty/ml | |
|---|---|---|
| Carfilzomib | 60 | mg |
| Dimethyl acetamide | 1000 | μL |

### Procedure:

1. 80% amount of required dimethyl acetamide was taken in clean vessel
2. Added required quantity of carfilzomib in step 1 at 2-8° C. and dissolved it with mechanical stirrer.
3. Made up the final batch size with required amount of dimethyl acetamide and mix it well at 2-8° C.
4. Filtered the bulk solution using 0.2-micron PTFE filter.
5. Filled in amber colour vial, followed by nitrogen blanketing and sealed it properly with rubber stopper.
6. The vials were kept on thermal stability at 25° C. /60% RH, 2-8° C. and 40° C./75% RH.

TABLE 7
Stability data of injectable composition before mixing of acidifying agent

| | | 25° C./60 | 25° C./60 | | | 40° C./75 | 40° C./75 |
|---|---|---|---|---|---|---|---|
| | | RH | RH | 2-8° C./ | 2-8° C./ | RH | RH |
| | Initial | 15 Days | 3M | 1M | 3M | 15 Days | 1M |
| Description | Complies | Complies | Complies | Complies | Complies | Complies | Complies |
| Related | | | | | | | |
| Substances | | | | | | | |
| Diol | ND | ND | ND | 0.01% | ND | 0.01% | 0.01% |
| Impurity | | | | | | | |
| N-Oxide | 0.01% | 0.01% | 0.02% | 0.02% | 0.01% | 0.03% | 0.05% |
| Impurity | | | | | | | |
| Chloro | 0.01% | 0.02% | 0.01% | 0.01% | ND | 0.07% | 0.03% |
| impurity | | | | | | | |
| Any other | 0.05% | 0.07% | 0.07% | 0.06% | 0.04% | 0.07% | 0.09% |
| individual | | | | | | | |
| impurity | | | | | | | |
| Total | 0.13% | 0.23% | 0.29% | 0.18% | 0.18% | 0.35% | 0.46% |
| Impurities | | | | | | | |

*ND = Not detected

TABLE 8
Procedure for mixing acidifying agent
to ready to dilute composition

| | | | | Final |
|---|---|---|---|---|
| Product Vial | Diluent vial | Volume of | Volume of | concentration |
| (Vial 1) | (Vial 2) | Vial 1 | Vial 2 | of API |
| 60 mg/mL | 90 microliter | 1 ml | 0.5 ml | 40 mg/mL |
| product | OPA in (50:50 | | | |
| Example 3 | water for | | | |
| | injection, USP | | | |
| | and dehydrated | | | |
| | ethanol) | | | |

1) Aseptically mixed the vial 1 and vial 2 as per the table mentioned above.
2) Gently swirled and/or inverted the vial slowly for about 1 minute.
3) Visually inspected for particulate matter and discoloration prior to mixing. The diluted product should be a clear, colorless solution and should not be mixed with infusion agent if any discoloration or particulate matter is observed.
4) The mixed product solution was diluted with sterile water for injection at the concentration of 0.5 mg/ml and 1.5 mg/ml and perform the solution stability at the 25° C. and 2-8° C.

TABLE 9
Stability data of injectable composition after mixing of acidifying agent at
concentration of 0.5 mg/ml and 1.5 mg/ml

| | 0.5 mg/ml | | | 1.5 mg/ml | | |
|---|---|---|---|---|---|---|
| Test parameters | Initial | 25° C./4 Hr | 2-8° C./24 Hr | Initial | 25° C./4 Hr | 2-8° C./24 Hr |
| Description | Complies | Complies | Complies | Complies | Complies | Complies |
| pH | 2.48 | 2.46 | 2.49 | 2.39 | 2.32 | 2.32 |
| Osmolarity | 135 | 136 | 136 | 387 | 385 | 389 |
| (msOm/kg) | | | | | | |
| Light | 99.132 | 99.397 | 99.684 | 98.680 | 99.109 | 99.214 |
| Transmittance % | | | | | | |
| Absorbance | 0.005 | 0.008 | 0.006 | 0.013 | 0.010 | 0.010 |
| Carfilzomib Assay | 96.80% | 95.6% | 95.8% | 90.30% | 89.7% | 90.5% |
| Diol Impurity | 0.11% | 0.15% | 0.16% | 0.13% | 0.13% | 0.16% |
| Any other single | 0.12% | 0.09% | 0.10% | 0.12% | 0.13% | 0.10% |
| individual max | | | | | | |
| impurities | | | | | | |
| Total Impurities | 0.36% | 0.37% | 0.45% | 0.44% | 0.46% | 0.53% |

### EAMPLE 11

### Carfilzomib Formulation Preparation

| Ingredients (Batch number) | APPL-006/01/062 | |
|---|---|---|
| Carfilzomib | 10 | mg |
| N N DMA | 250 | mg |
| Polysorbate 80 | 300 | μl |
| Alpha-Tocopherol | 0.2 | mg |
| Water for injection | 30 | mg |

| | |
|---|---|
| Super fined Propylene glycol | Qs to 1 ml |

### Procedure:

1. Standard quantity of N, N-Dimethylacetamide was taken in a manufacturing vessel at 2-8° C. temperature.
2. Weighed batch quantity of Alpha Tocopherol was added under continuous stirring until uniform solution obtained.
3. Weighed batch quantity of Carfilzomib was added under continuous stirring until complete dissolved.
4. Weighed batch quantity of water for injection was added under continuous stirring until uniform solution obtained.
5. Weighed batch quantity of Polysorbate 80 was added under continuous stirring until uniform solution obtained.
6. Final volume was made-up by Super refined PG and stirred until uniform solution was obtained.
7. The bulk was filtered through 0.22 μ PTFE filter and filled into vial.

### EXAMPLE 12

### Carfilzomib Formulation Preparation

| Ingredients (Batch number) | APPL-006/01/063 | |
|---|---|---|
| Carfilzomib | 10 | mg |
| Polysorbate 80 | 300 | μl |
| Alpha-Tocopherol | 0.2 | mg |
| Ethanol (Dehydrated Alcohol) | 500 | |
| Water for injection | 30 | mg |

| | |
|---|---|
| Super fined Propylene glycol | Qs to 1 ml |

### Procedure:

1. Standard quantity of ethanol was taken in a manufacturing vessel at 2-8° C. temperature.
2. Weighed batch quantity of Alpha Tocopherol was added under continuous stirring until uniform solution obtained.
3. Weighed batch quantity of Carfilzomib was added under continuous stirring until complete dissolved.
4. Weighed batch quantity of water for injection was added under continuous stirring until uniform solution obtained.
5. Weighed batch quantity of Polysorbate 80 was added under continuous stirring until uniform solution obtained.
6. Final volume was made-up by Super refined PG and stirred until uniform solution was obtained.
7. The bulk was filtered through 0.22 μ PTFE filter and filled into vial

### EXAMPLE 13

### Carfilzomib Formulation Preparation

| Ingredients (Batch number) | APPL-006/01/066C | |
|---|---|---|
| Carfilzomib | 10 | mg |
| Polysorbate 80 | 300 | mg |
| Alpha-Tocopherol | 0.2 | mg |
| Ortho phosphoric acid (OPA) | QS to pH 5-6 | |
| Dimethyl acetamide | 250 | mg |
| Water for injection | 30 | mg |

| | |
|---|---|
| Super refined Propylene glycol | QS to 1 ml |

### Procedure:

1. Standard quantity of dimethyl acetamide was taken in a manufacturing vessel at 2-8° C. temperature.
2. Weighed batch quantity of Alpha Tocopherol was added under continuous stirring until uniform solution obtained.
3. Weighed batch quantity of Polysorbate 80 was added under continuous stirring until complete dissolved.
4. Weighed batch quantity of water for injection was added under continuous stirring until uniform solution obtained.
5. Weighed batch quantity of Super refined PG was added under continuous stirring until uniform solution obtained.
6. Weighed batch quantity of Carfilzomib was added under continuous stirring until uniform solution obtained.
7. Final volume was made-up by Super refined PG and stirred until uniform solution was obtained.
8. The bulk was filtered through 0.22 μ PTFE filter and filled into vial

### EXAMPLE 14

### Carfilzomib Formulation Preparation

| Ingredients (Batch number) | APPL-006/01/067 | |
|---|---|---|
| Carfilzomib | 10 | mg |
| Polysorbate 80 | 300 | mg |
| Alpha-Tocopherol | 0.2 | mg |
| Ortho phosphoric acid (OPA) | QS to pH 5-6 | |
| Water for injection | 30 | mg |
| Super refined Propylene glycol | QS to 1 ml | |
| Ethanol | 400 | mg |

### Procedure:

1. Standard quantity of Ethanol was taken in a manufacturing vessel at 2-8° C. temperature.
2. Weighed batch quantity of Alpha Tocopheral was added under continuous stirring until uniform solution obtained.
3. Weighed batch quantity of Polysorbate 80 was added under continuous stirring until complete dissolved.
4. Weighed batch quantity of water for injection was added under continuous stirring until uniform solution obtained.
5. Weighed batch quantity of Super refined PG was added under continuous stirring until uniform solution obtained.
6. Weighed batch quantity of Carfilzomib was added under continuous stirring until uniform solution obtained.
7. Final volume make-up by Super refined PG and stir until uniform solution obtained.
8. The bulk was filtered through 0.22 μ PTFE filter and filled into vial.

### EXAMPLE 15

### Carfilzomib Formulation Preparation

| Ingredients (Batch number) | APPL-006/01/102 |
|---|---|
| Carfilzomib | 60 mg |
| N,N DMA | QS to 1 mL |

### Procedure:

1. 90% of N, N-Dimethylacetamide was taken in a manufacturing vessel at 2-8° C. temperature.
2. Weighed batch quantity of Carfilzomib was added under continuous stirring until uniform solution obtained.
3. Final volume was made-up by N, N-Dimethylacetamide and stirred until uniform solution was obtained.
4. Filter the bulk through 0.22 μ PTFE filter and filled in vial.

### EXAMPLE 16

### Carfilzomib Formulation Preparation

| Ingredients (Batch number) | APPL-006/01/103 | |
|---|---|---|
| Carfilzomib | 30 | mg |
| N,N DMA | 500 | μl |

| | |
|---|---|
| Ethanol | QS to 1 mL |

### Procedure:

1. Weighed batch quantity of N, N-Dimethylacetamide was taken in a manufacturing vessel at 2-8° C. temperature.
2. Weighed batch quantity of Carfilzomib was added under continuous stirring until uniform solution obtained.
3. Final volume was made-up by Ethanol and stirred until uniform solution was obtained.
4. The bulk was filtered through 0.22 μ PTFE filter and filled in vial.

### EXAMPLE 17

### Carfilzomib Formulation Preparation

| | |
|---|---|
| Batch Size: | 50 mL |
| Batch No. | APPL-006/01/105 |

| Ingredients | Quantity (mg/mL) |
|---|---|
| Carfilzomib | 60.0 |
| N,N-Dimethylacetamide | Q.S. to mL |

### Procedure:

1. 80% of N, N-Dimethylacetamide was taken in a manufacturing vessel at 2-8° C. temperature.
2. Weighed batch quantity of Carfilzomib was added under continuous stirring until uniform solution obtained.
3. Final volume was made-up by N, N-Dimethylacetamide and stirred until uniform solution was obtained.
4. The bulk was filtered through 0.22 μ PTFE filter and filled in vial.

TABLE 10
Thermal stability and analytical results of APPL-006/01/105

| | Time point | | | | |
|---|---|---|---|---|---|
| | | 40 ± 2° C./ | | | 25 ± 2° C./ |
| | Initial | 75 ± 5% RH | | | 60 ± 5% RH |
| | Condition | | | | |
| | | 1 M | 2 M | 3 M | 3 M |
| Description | Clear | Clear | Clear | Clear | Clear |
| | solution | solution | solution | solution | solution |
| Assay of Carfilzomib | 101.2% | 100.5% | NA | 102.5% | 101.4% |
| Related substance | | | | | |
| Acid Impurity | ND | ND | ND | ND | ND |
| Chloro Impurity | ND | 0.02% | ND | ND | ND |
| Diol Impurity | ND | ND | ND | ND | ND |
| Diastereomer | ND | ND | ND | ND | ND |
| Impurity | | | | | |
| N-Oxide Impurity | ND | 0.06% | 0.07% | 0.11% | ND |
| Highest individual | 0.04% | 0.15% | 0.07% | 0.11% | 0.05% |
| unspecified impurity | | | | | |
| Total impurity | 0.07% | 0.38% | 0.32% | 0.36% | 0.11% |

NMT: Not more than,
ND—Not detected

### EXAMPLE 18

### Carfilzomib Formulation Preparation

| | |
|---|---|
| Batch Size: | 50 mL |
| Batch No. | APPL-006/01/106 |

| Ingredients | Quantity (mg/mL) |
|---|---|
| Carfilzomib | 30.0 |
| N,N-Dimethylacetamide | Q.S. to mL |
| Ethanol | 500.0 μL |

### Procedure:

1. Weighed batch quantity of N, N-Dimethylacetamide was taken in a manufacturing vessel at 2-8° C. temperature.
2. Weighed batch quantity of Carfilzomib was added under continuous stirring until uniform solution obtained.
3. Final volume was made-up by ethanol and stirred until uniform solution was obtained.
4. The bulk was filtered through 0.22 μ PTFE filter and filled in vial.

TABLE 11
Thermal stability and analytical results of APPL-006/01/106

| | Time point | | | | |
|---|---|---|---|---|---|
| | | 40 ± 2° C./ | | | 25 ± 2° C./ |
| | Initial | 75 ± 5% RH | | | 60 ± 5% RH |
| | Condition | | | | |
| | | 1 M | 2 M | 3 M | 3 M |
| Description | Clear | Clear | Clear | Clear | Clear |
| | solution | solution | solution | solution | solution |
| Assay of Carfilzomib | 105.8% | 103.5% | NA | 102.6% | 102.6% |
| Related substance | | | | | |
| Acid Impurity | ND | ND | ND | 0.03% | ND |
| Chloro Impurity | 0.01% | 0.11% | 0.05% | 0.23% | 0.04% |
| Diol Impurity | ND | ND | 0.14% | 0.22% | ND |
| Diastereomer | ND | ND | ND | ND | ND |
| Impurity | | | | | |
| N-Oxide Impurity | 0.01% | 0.01% | 0.05% | 0.21% | 0.05% |
| Highest individual | 0.06% | 0.18% | 0.24% | 0.48% | 0.1% |
| unspecified impurity | | | | | |
| Total impurity | 0.13% | 1.05% | 1.55% | 2.40% | 0.19% |

NMT: Not more than,
ND—Not detected

1.-34. (canceled)

35. A room temperature stable ready to dilute injectable formulation comprising carfilzomib or its pharmaceutically acceptable derivatives thereof and one or more solvents; wherein the formulation contains no more than 6% total impurities when stored for at least three months.

36. The room temperature stable ready to dilute injectable formulation according to claim 35, wherein the concentrations of carfilzomib or its pharmaceutically acceptable derivatives thereof are in the range from about 10 mg/mL to about 100 mg/mL.

37. The room temperature stable ready to dilute injectable formulation according to claim 36, wherein the concentrations of carfilzomib or its pharmaceutically acceptable derivatives thereof are in the range from about 10 mg/mL to about 60 mg/mL.

38. The room temperature stable ready to dilute injectable formulation according to claim 35 and optionally one or more pharmaceutically acceptable excipients selected from antioxidant, buffer, preservative, and surfactant.

39. The room temperature stable ready to dilute injectable formulation according to claim 35, wherein the formulation contains no more than 6% total impurities when stored for at least six months.

40. The room temperature stable ready to dilute injectable formulation according to claim 35, wherein the one or more solvents is selected from ethanol, isopropyl alcohol, benzyl alcohol, propylene glycol, polyethylene glycol, glycerol, dimethylacetamide, N-methylpyrrolidone, dimethylsulfoxide (DMSO), diethylene glycol monoethyl ethers, caprylocaproyl polyoxyl-8 glycerides, glycofurol, or mixtures thereof.

41. The room temperature stable ready to dilute injectable formulation according to claim 40, wherein the solvent is mixed in suitable ratio (w/w) with carfilzomib or its pharmaceutically acceptable derivatives thereof which is from about 100:1 to 8:1.

42. The room temperature stable ready to dilute injectable formulation according to claim 38, wherein the antioxidant is selected from butylated hydroxytoluene, butylated hydroxy anisole, propyl gallate, and C.-tocopherol, DL-tocopherol, C-tocopherol acetate, C.-tocopherol Tocopherol Polyethylene Glycol Succinate (Vitamin E TPGS), L-cysteine ascorbyl palmitate thioglycolic acid, sodium metabisulfite (SMBS), ascorbic acid, sodium formaldehyde sulfoxylate, or hydrophilic antioxidants, including sodium EDTA and thioglycerol.

43. The room temperature stable ready to dilute injectable formulation according to claim 42, wherein the antioxidant is present in concentration from about 0.005% to about 5% weight/weight of the total composition.

44. The room temperature stable ready to dilute injectable formulation according to claim 35, wherein the formulation is stored at 25° C. and 60% relative humidity or at 40° C. and 75% relative humidity.

45. The room temperature stable ready to dilute injectable formulation according to claim 35, wherein the formulation contains no more than 5%, no more than 4%, no more than 3%, no more than 2%, no more than 1.5%, no more than 1% or no more than 0.5% of total impurities upon storage period.

46. A method for treating patients with relapsed or refractory multiple myeloma which comprises administering a room temperature stable ready to dilute injectable formulation of carfilzomib or its pharmaceutically acceptable derivatives thereof; and one or more solvents; wherein the formulation contains no more than 6% total impurities when stored for at least three months.

47. The method for treating patients with relapsed or refractory multiple myeloma according to claim 46, wherein the ready to dilute injectable formulation is stable when stored for at least six month.

48. A method of administering a reconstituted ready to dilute formulation comprising mixing of a component 1 and a component 2 to form the reconstituted ready to dilute formulation followed by diluting the reconstituted ready to dilute formulation with an infusion media; wherein component 1 comprises a room temperature stable ready to dilute formulation of carfilzomib or its pharmaceutically acceptable derivatives thereof and contains no more than 6% of total impurities when stored for at least three months; and component 2 is an acidifying agent.

49. The method of administering reconstituted ready to dilute formulation according to claim 48 wherein component 1 and component 2 are in the range from about 10 mg/mL to about 60 mg/m

50. The method of administering reconstituted ready to dilute formulation according to claim 48, wherein the acidifying agent is mixed with carfilzomib or its pharmaceutically acceptable derivatives thereof in a suitable ratio (w/w) selected from about 1:50 to about 1:1.

51. The method of administering reconstituted ready to dilute formulation according to claim 48, wherein the component 1 is stored for at least three months at 25° C. and 60% relative humidity or at 40° C. and 75% relative humidity.

52. The method of administering reconstituted ready to dilute formulation according to claim 48, wherein the component 1 comprises room temperature stable ready to dilute formulation of carfilzomib or its pharmaceutically acceptable derivatives and contains no more than 6% of total impurities when stored for at least six months.

53. The method of administering reconstituted ready to dilute formulation according to claim 48, wherein component 1 contains no more than 5%, no more than 4%, no more than 3%, no more than 2%, no more than 1.5%, no more than 1% or no more than 0.5% of total impurities upon storage period.

54. The method of administering reconstituted ready to dilute formulation according to claim 48 wherein the acidifying agent is selected from citric acid, acetic acid, maleic acid, phosphoric acid, succinic acid, tartaric acid, ascorbic acid, benzene sulfonic acid, oxalic acid, fumaric acid, ortho phosphoric acid, gluconic acid, malic acid, methane sulfonic acid, p-toluenesulfonic acid, naphthalene sulfonic acid, lacturonic acids, lactic acid, lactobionic acid, edetic acid, gentisic acid, meta phosphoric acid, nitric acid, pentetic acid, glycolic acid or mixtures thereof.

55. A method for treating patients with relapsed or refractory multiple myeloma which method comprises administering a reconstituted ready to dilute formulation comprising mixing of component 1 and component 2 to form reconstituted ready to dilute formulation followed by diluting the reconstituted ready to dilute formulation with an infusion media; wherein component 1 is a room temperature stable ready to dilute parenteral formulation of carfilzomib or its pharmaceutically acceptable derivatives and component 2 is an acidifying agent and wherein the reconstituted ready to dilute composition has total impurity of not more than 2% up to 6 hours after mixing when the mixture is stored at room temperature.

56. The method for treating patients with relapsed or refractory multiple myeloma according to claim 55, wherein the reconstituted ready to dilute composition has total impurity not more than 1% up to 6 hours, not more than 2% up to 5 hours, not more than 1% up to 5 hours, not more than 2% up to 4 hours or not more than 1% up to 4 hours after mixing when the mixture is stored at room temperature.
